(19) United States
(12) Patent Application Publication
(10) Pub. No.: US 2005/0180940 A1
(43) Pub. Date: Aug. 18, 2005

(54) Protect against hair loss

(76) Inventor: Jeffrey Paul Puma, Wantagh, NY (US)

Correspondence Address: JEFFREY PUMA, 44 TWIN LN, WANTAGH, NY, 11793, US

(21) Appl. No.: 10/876,953

(22) Filed: Jun. 25, 2004

Related U.S. Application Data
(63) Continuation-in-part of application No. 10/779,947, filed on Feb. 17, 2004, now abandoned.

(51) Int. Cl. A61K 7/06; A61K 7/075; A61K 7/08

(52) U.S. Cl. 424/70.24

(57) ABSTRACT

A topical composition to protect against hair loss comprising of Sodium diisopropyl naphthalene sulfonate, Butylene glycol, Hampshire N-acyl LED3A, Hamposyl M-30, Hamposyl TC Glutamate. In a preferred embodiment, the composition contains the primary active ingredient sodium diisopropyl naphthalene sulfonate or alkyl naphthalene sulfonate and is commercially available from Cytec Industries under the product name of Aerosol OS Surfactant. The Aerosol OS Surfactant is in the chemical family of alkyl aryl sulfonate. In application as a scalp shampoo for follicle treatment the Aerosol OS Surfactant is an active ingredient of sulfur compounds with anti-inflammatory effects to protect against hair loss. The Aerosol OS can be comprised with any surfactant, chelate or inert carrier, etc. In particular, the composition unblocks the follicle, releases entrapped hair and oxidizes proteins into metabolism allowing the dermal papilla the movement to rise and decline during follicle assimilation.

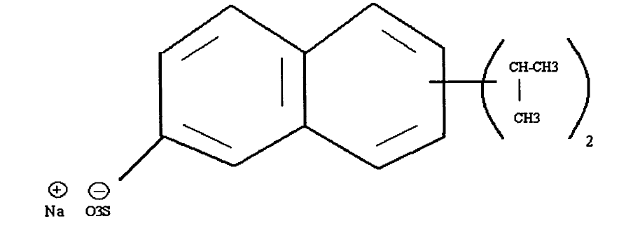

This application is a Continuation in part (CIP) of application Ser. No. 10/779,947, filing date Feb. 17, 2004, Composition to protect against hair loss.

### REFERENCES CITED

### U.S. Patent Documents

| | | | |
|---|---|---|---|
| 6,645,477 | November, 2003 | Jarrousse, et al. | 424/70.1 |
| 6,358,541 | March, 2002 | Goodman | 424/727 |
| 6,139,828 | October, 2000 | McCullough | 424/70.24 |
| 5,690,948 | November, 1997 | McCook, et al. | 424/401 |
| 5,641,480 | June, 1997 | Vermeer | 424/70.24 |
| 5,620,980 | May, 1997 | Samour | 514/256 |
| 5,026,553 | June, 1991 | Swinney | 424/757 |
| 4,588,750 | May, 1986 | Boris | 514/765 |

### TECHNICAL FIELD

The present invention relates to a topical solution applied to the scalp that is suitable to protect against hair loss. A uniform resist in the wall of a follicle will provide a zero tolerance of laminate formed by follicle assimilation. A blocked hair follicle can trap a hair especially, during the catogen and telogen phase when the presence or movement of the hair decreases or stops. Also, an accumulation of lipid mixes, di-hydrotestosterone (DHT), sebum and sweat films can be detrimental and cause damage to the follicle and hair root, which can lead to hair loss. The present invention unblocks the follicle, releases entrapped hair and oxidize proteins into metabolism without the use of drugs and risk of carcinogens.

### BACKGROUND OF THE INVENTION

There are countless scalp treatment shampoos. Typically, they have some ability to help the hair and scalp. While most of these compositions serve the purpose, they primarily contain nutrients, herbs, and now, extracts from exotic herb roots. Recently, many topical compositions added to their formula high potent nutrients derived from a secondary enzymatic process. Most of the vitamins and minerals the body needs can be found in an ordinary balanced diet. Vitamins and supplements are generally recommended when there is a pregnancy or deficiency in the diet, particularly for the elderly or poor, to be taken internally where they have intended purpose. Any superficial application, such as being applied externally, does not contribute to their reaction in metabolism where they are most beneficial. The problem with topical compositions combined with nutrients or bionutrients is their inability to react as effectively as when taken internally.

Another problem is scalp compositions containing an antibacterial. Many provide minor results since the surfactant ingredient used is common among other topical solutions which generally have disinfecting properties. The disinfecting of the scalp or follicle helps control bacteria or inflammation, only. The follicle is disinfected, yet laminate remains. Moreover, is the hair care system consisting of a variety of products. The hair care system recommends the user to apply a pre-rinse treatment to remove hair styling residue, a shampoo treatment to clean the hair, a scalp treatment to clean the scalp surface residue, an antimicrobial treatment to control bacteria, a bionutrient treatment to nourish the hair, and hair conditioner treatment to soften or help damaged hair. Next, styling products are applied to achieve a desired style of the hair. Finally, the hair care system recommends an oral supplement vitamin and apply their overnight treatment to your scalp.

The main thrust of the problem when applying multiple products or their extensive use is to the hair, scalp and follicle. The duplicitous or extensive treatment may cause scalp irritation, damaged hair, or may result in acute hair root shock due to over exposure of chemical treatments.

In these respects, the present invention departs from the conventional concepts of the prior art and in doing so, provides a scalp composition treatment suitable to protect against hair loss by unblocking the follicle, releasing entrapped hair and oxidizing proteins into metabolism.

### THE PRIOR ART

U.S. Pat. No. 6,689,348, to Jeon discloses a composition for a method of treating hair loss with herbal extracts, diethanol or triethanol amine, diocytl sodium sulfosuccinate and benzoic acid. The composition as stated in claim 2 and 3, is fermented in a ultrasonic fermenting apparatus between 12 to about 24 hours. The present invention stated herein departs from the prior art in the ingredients and the method of comprising said ingredients.

U.S. Pat. No. 5,767,104, to Bar-Shalom, et al., discloses a method of treating or preventing baldness with sulfated saccharine. The prior art as stated in Example 14: Effect of a treatment with sucralfate on common male-pattern baldness; states the preparation was used in two daily topical applications by a 40-year-old male who had a normal, common male-pattern baldness. After 3-4 weeks, there was appearance of hair in the otherwise bald lateral-frontal areas in the scalp. The present invention stated herein departs from the prior art in the ingredients and the amount of daily treatments.

U.S. Pat. No. 5,597,575, to Breitbarth discloses a topical treatment for the scalp. Claim 1 states a composition for stimulating and inducing hair growth. Claim 2 states the ingredients consisting of vitamin D.sub 3 component attached to said micro particles via charged ionic groups. Claim 4 states a composition comprising of vitamin D.sub.3, non-metallic particles, a surfactant of 3.5%-6.0% by volume, a preservative, and aloe. Claim 9 specifically identifies the surfactant as Tween 20. The prior art and its stated ingredients have no similarity to the present invention. In the prior art claim 8-12 describe a method to prepare its composition. In the prior art claim 13, 14 and 15 states a method of treating a subject to stimulate and induce hair growth is by applying the effective amount of the composition to the scalp and massaging the drops of the composition into the scalp for a period of five minutes once a day. In conclusion, the present invention stated herein departs from the prior art as a topical composition in ingredients, method of preparation and directions for use.

Other body treatment topical compositions for the scalp to treat hair loss and baldness are set forth in the following U.S. Patents: Green, U.S. Pat. No. 4,529,587; Mathews, et al., U.S. Pat. No. 4,814,351; Tragar, U.S. Pat. No. 5,043,162; Goldman, U.S. Pat. No. 5,407,944; DiTucci, U.S. Pat. No. 5,674,510; Dufetel et al., U.S. Pat. No. 5,760,043; Bradbury et al., U.S. Pat. No. 6,451,777.

### SUMMARY OF THE INVENTION

The object of the present invention is a scalp treatment suitable to protect against hair loss. This claim is accomplished, in accordance with the present invention by providing a liquid composition of anionic surfactants and a chelate. The composition develops to a critical miscelle concentration (CMC) and essentially the optimum of activity. The force acting on the surface of the liquid is minimized as the surface tension is reduced providing a zero tolerance of follicle assimilation laminate during the hair growth cycle.

The present invention is a rheology of agents with properties that strongly absorb along the entire hair shaft and follicle providing a boost of foam in the presence of lipid oils. The addition of a chelate agent, combines the chelate power with the potential to chelate transition metals which catalyze lipid per oxidation. The main ingredient, sodium diisopropyl naphthalene sulfonate is a highly effective surface active agent, even more effective, once the other agents are precipitated or decomposed. The sodium diisopropyl naphthalene sulfonate is a unique ingredient. According to Cytec Industries brochures and literature, the manufacturer makes no reference or claim to document its use in a shampoo, cosmetic or hair care application and moreover, the present invention comprising sodium diisopropyl naphthalene sulfonate in a scalp shampoo was discovered independently without manufacturers' or supplier's recommended use.

To protect against hair loss, the present invention as applied to the scalp skin, has active ingredients to eliminate follicle laminate. Moreover, the separating of the acidic containing impurities and protein decay occurs particularly, at the mouth of the follicle. Within a blocked follicle, the population of normal bacteria increases dramatically. A blocked follicle can trap the hair and other bacteria inside, too. Proteins that are vital in the anagen phase are spent fighting bacteria. Also, a blocked follicle can produce depression or deactivation of the hair bulb. While the presence of the hair and its movements may help prevent the hair follicle from becoming blocked, this usually occurs during the anagen phase. The risk of a blocked follicle, entrapped hair and protein loss occur in the catagen resting or the telogen regression phase. The excess shedding of hair and hair loss may be caused by any of the above circumstances.

The alkyl is selected from the group consisting of ethyl, hexyl, heptyl, isobutyl, isopropyl, methyl, n-butyl, octyl, pentyl, propyl, and tertbutyl. The alkyl has from 1 to 10 carbon atoms and can have 9 to 10 carbon atoms, or can have 5 to 7 carbon atoms, and preferably has 1 to 3 carbon atoms, and most preferably has 3 carbon atoms. The sodium diisopropyl naphthalene sulfonate can be a solid or liquid and substituted with sodium diisobutyl naphthalene sulfonate, alkyl aryl naphthalene sulfonates or dodecyl diphenyl oxide sulfonate.

The present invention can be used in a variety of methods. It can be added to hair or scalp products. It can be comprised to a solid or aqueous form and used as a powder, gel, rinse, lotion, cream, tonic, spray, or overnight treatment.

The composition can have ingredients added such as preservatives, antiseptics, nutrients, vitamins, herbs, fragrances, dyes and detergents.

While only a single embodiment of the present invention has been shown and described, it is to be understood that many changes and modifications may be made thereunto, without departing from the spirit and scope of the invention as defined by the appended claims.

### OTHER REFERENCES

The Chemistry and Manufacture of Cosmetics: M. Schlossman, V. II, III, 2001.

The Trichological Society: Moore, Surfactants and Shampoos, 2001.

Hamposyl Surfactants Sixth Edition: Applications and Analytic Methods, 2002.

Cedar Concepts: Surface Active Organic Chemicals, 2002.

Cosmetics Unmasked: Antczak, Potentially Harmful Chemicals, 2002.

The American Oil Chemist Society: Crandall, Surfactant Trends, V. 14, No. 5, 2003.

Cytec Industries Inc: Nimal Jayasuriya, Structure of Aerosol OS, 2004.

The Thicket Hair Clinic: F. Wittenstein, 2004.

1. A method of manufacturing a composition suitable for treatment to protect against hair loss on a human scalp, the composition having the following constituents, by percentage of liquid volume of total ingredients:
a) water, from 0.005% to 99.5%,
b) Sodium diisopropyl naphthalene sulfonate, from 0.005% to 99.9%,
c) Butylene glycol, from 0.005% to 50%,
d) N-acyl LED3A, from 0.005% to 30%,
e) Hamposyl M-30, from 0.005% to 30%,
f) Hamposyl TC Glutamate, from 0.005% to 30%.

2. A method of treatment;
a) apply the topical solution to the scalp one time daily;
b) in an amount from 1 to 3 milliliters,
c) massage on bald and thinning areas for 1 minute,
d) spread to the entire scalp,
e) leave on scalp for 1 minute,
f) rinse scalp with water,
g) apply for no less than 90 days.
